(19) United States
(12) Patent Application Publication
(10) Pub. No.: US 2025/0036087 A1
(43) Pub. Date: Jan. 30, 2025

(54) NOVEL CLOCK/TIMER/METER TO INDICATE THE NEED TO REPOSITION A PATIENT AND THE LIKE

(71) Applicants: John Alexander, Minneapolis, MN (US); Kari Beth Miller, Minneapolis, MN (US)

(72) Inventors: John Alexander, Minneapolis, MN (US); Kari Beth Miller, Minneapolis, MN (US)

(21) Appl. No.: 18/782,797

(22) Filed: Jul. 24, 2024

Related U.S. Application Data
(60) Provisional application No. 63/528,449, filed on Jul. 24, 2023.

(51) Int. Cl. G04G 13/02; A61B 5/00

(52) U.S. Cl. CPC G04G 13/021; A61B 5/447; A61B 5/742; A61B 5/746; A61B 5/7475

(57) ABSTRACT

A visual electrical, time management device incorporating a display which indicates the time lapse between resets, and alerts clinical staff to the relative urgency to act for a patient unable to act for his or her own self. The invention is used to improve awareness and compliance for repositioning protocol for clinical staff or caregivers that a patient should be turned, moved, or repositioned to avoid DUs associated with long periods of time in the same position in bed. The device measures the time elapsed since last reset indicating how long the patient has been in the current position. The device “face” provides an easy to identify and understand visual or audio cues to how much time has lapsed and how much time is left before the patient may experience injury. The caregiver resets the device easily by activating the face directly, by voice, or other electronic method.

### CROSS-REFERENCE TO RELATED APPLICATION

The present application claims the benefit of U.S. Provisional Application No. 63/528,449, filed Jul. 24, 2023, which is hereby incorporated by reference in its entirety.

### FIELD OF TECHNICAL INVENTION

The present invention relates to a novel device which provides a visual, electronic, or other form of reminder to caregivers for the need to act for a patient unable to act for himself. More particularly, the invention relates to the novel method of displaying a graphic representation of the passage of time between events. This invention is a critical tool in the management and prevention of life threatening Decubitus Ulcers (“DU”s). The use of this device will assist caretakers, in the home or clinical setting, an easy way to identify when the next event and/or task is required. When used appropriately and consistently, this tool will improve adherence to time sensitive treatments and protocols by the caretaker or patient.

### BACKGROUND

Caretakers of bedridden or sensory impaired patients, require an indication of the amount of time the patient has been one position. If a sensory impaired individual remains in one position for a prolonged period of time, injury to the skin will develop called a Decubitis Ulcer (DU). DU's also called bedsores, or pressure ulcers, are injuries to skin and underlying tissues (including muscle and bone) resulting from lack of blood supply to a prominent area due to prolonged pressure on the skin.

Prolonged pressure in one area impairs circulation resulting in tissue compromise and cellular death. Tissue compromise can be at the surface area of the skin or may involve the deeper tissue including fat, muscle and bone. The area most at risk for DUs is the skin that covers bony areas of the body, such as the heels, ankles, hips, tailbone, elbows, shoulders, and or ears.

People most at risk for DUs have underlying medical conditions or contributing factors that limit their ability to change positions on their own, have impaired sensory perception, or cause them to spend most of their time in a bed or chair.

DUs can begin to develop within hours and progress over time when the skin is subjected to pressure. Some ulcers heal with treatment, but others never heal completely. Moreover, DUs can increase the risk for life threatening infections which require higher and more expensive levels of care. The cost of DUs to the US healthcare system is estimated to be $26.8 billion (2018) per year.

The primary intervention for the treatment and prevention of DU must include frequent repositioning in order to establish blood flow to the area and promote healing. Repositioning of the patient allows for impaired circulation at the pressure points to be relieved and normal circulation to that area can be reestablished and allow for healing.

The more often a patient is turned or repositioned, the lower the risk for injury to the skin in the vulnerable pressure points.

Without repositioning, the skin will continue to sustain injury to the impaired areas and result in impaired skin integrity and increase the risk of life-threatening infections. Nursing home and hospital standard protocol calls for the treatment and prevention of DU's require the repositioning a patient at least once every two hours, twenty four hours a day. Professional staff and home caretakers often fail to adhere to this protocol despite current processes to support this treatment. Caretakers require a reminder to alert them of time passed since the last repositioning. This can be a challenge when there are multiple caretakers and/or they are busy doing other tasks. Without a reminder, patients are left for a position for too long and develop DUs or exacerbate those they already have.

There are no common, affordable, easy to use, and reliable automated time-tracking devices for this use on the market today. Many caregivers may document the last time a patient was turned and add 2 hours to indicate when the next “turn” event is due. This method fails for a variety of reasons: That information is not easily shared with others, relies on someone to calculate time intervals, and requires the manual task to write the information down. Others try to set a specific schedule with paper time clock with markings to say when the next turn is due. However, this does not display the amount of time lapse since last turn which is critical in determining when the next “turn” event should occur. Either of the current methods fail if the patient is turned early or a turn time is missed. Still others create lists or personal reminders on cell phones or paper to prompt a turn. Bed alarms and other alerts are disruptive and cause sleep disturbances and often causing the patient to fall. All current solutions have proven to be inadequate solutions to avoid DUs from developing, or decrease the cost to the healthcare system.

DUs cost the American healthcare system $26.8 Billion a year (2018 data). DUs are the number one patient harm incurred in a care facility: about 2.5 million people a year suffer from DUs, and an estimated 60,000 die from the poor outcomes resulting from DUs. If DUs were cancer, the condition would kill more people than every type of cancer, except for lung cancer.

### BRIEF SUMMARY OF THE INVENTION

Caregivers require a reminder to alert them of when a patient needs to be repositioned or any other care needs to be given, i.e., medication needs to be given. This device will provide an easy to use, low cost time management solution for caregivers to communicate when a turn, medication reminder, physical activity (non-turn) reminder, ADD time limits for children, or other event, last occurred and when it is time to reposition or provide other treatment/action again. Each time the patient is repositioned or provided according treatment, the caregiver resets, manually or otherwise “taps,” voice activates, etc. the device (timer), and the timer begins again.

The present invention is comprised of an electronically operated (e.g., battery powered, wired power, etc.) device 100 with an easily readable indicator or “clockface” type display 102 which provides a visual indication of the time when the device was last tapped. If the caretakers 104 taps the device every time a patient is turned, it becomes a common tool to communicate action performed and support the timeliness of a repositioning intervention. The current embodiment of the device 100 includes a “clockface” 102 which has a color and numeric display signaling caregivers to reposition the patient off pressure points since last repositioning event and indicate remaining time for optimal skincare needs.

The clockface 102 has a hand, or other indicator 106, which starts at the zero position and moves in a clockwise rotation toward the start and finishes in one revolution. Different time intervals can be predefined for the device 100 (e.g., clock); such as 30 minutes, 45 minutes, 60 minutes, 2 or 4 hours, etc. It should be understood that while particular time intervals have been exemplified herein, alternative amounts of time may be set. For example, while a clock/timer 100 may be set to countdown from a programmed time period/intervals, the clock/timer 100 is not limited to counting down from a specific time, per se. The time limit set or programmed may determine at what amount of time passing (e.g., portions, fractions, percentages, etc.) results in an allocation of what amount of time equates to each portion, fraction, percentage, etc. of the clock face 102 and the limit or interval in which may execute an alert, sound, notification, etc. The clockface 102 may be portioned into a variety of time management positions to indicate no action is necessary, warnings, time for action, etc. Indications may be based on the indicated time intervals, preset time intervals, or changed time intervals by the clinician. This flexibility is important depending on what action is to be taken at a particular time, the condition of the user, and many other various factors relative actions that may not have the same timing interval flexibilities.

For example, a particular time patient may need to be repositioned every 2 hours. As discussed in more detail below, the clockface 102 may be portioned into 3 sections, such that half 108 of the clockface 102 indicates green for 1 hour, a quarter 110 of the clockface 102 indicates yellow for 30 minutes, and a remaining quarter 112 of the clockface 102 indicates red for the remaining 30 minutes. In another example, a clinician 104 may program the device 100 to indicate repositioning of a patient every 6 hours, such that half 108 of the clockface 102 indicates green for 3 hours, a quarter 110 of the clockface 102 indicates yellow for 1.5 hours, and a remaining quarter 112 of the clockface 102 indicates red for the remaining 1.5 hours. It should be understood that, while exemplary portions of the clockface 102, based on repositioning (or other action) time intervals of a patient, have been described herein, alternative time intervals and portioned clockfaces based on those time intervals are contemplated.

In the current embodiment of the device 100, there may be three different portions or zones of the clockface 102. The green area 108 (first area the hand moves through) may indicate a desired amount of time and no action is required. The yellow area 110 (the second area the hand 106 may move through) may indicate time is close to expire and action is required soon. The red zone 112 (the final area) may indicate the action is required now (at or before the time is exceeded in that portion or zone). The device 100 may also signal to caretakers 104 via other devices by Bluetooth, a text message or another telecommunication mechanism.

The device stores data about the time interval between events, e.g. time of the turn, time since the last turn, indicates whether the time between turns exceeds the desired maximum time (for example, 2 hours). This data can be used to identify compliance to the treatment plan and potential outcomes and ultimately the cost and effectiveness of patient care.

Device Use: Once the specified patient event has occurred such as repositioning, the caregiver 104 taps (push and release) the device 100 by pushing anywhere on the face 114 (with a hand, fist, elbow, knee, or extension device, or with a voice prompt or other electronic mechanism) and the device 100 is reset back to zero. The hand 106 may move to the start point (or indicator begins at the start point) and the clock may immediately restart the timer to indicate time passing. When used to mark repositioning events, this will act as a documentation support and communication tool to display the amount of time a patient has been in the current position.

The advantages of this device 100 includes: easy to understand visual graphic and digital display regardless of the age, role, language or education level of the caregiver. It features a simple reset mechanism, is user friendly, efficient, consistent, and records patient repositioning data to assist staff in properly executing care, and improving the quality and cost of care.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

Having thus described various embodiments of the invention in general terms, reference will now be made in the accompanying drawings, which are not necessarily drawn to scale, and wherein:

FIG. 1 illustrates a front perspective view of one example of the device.

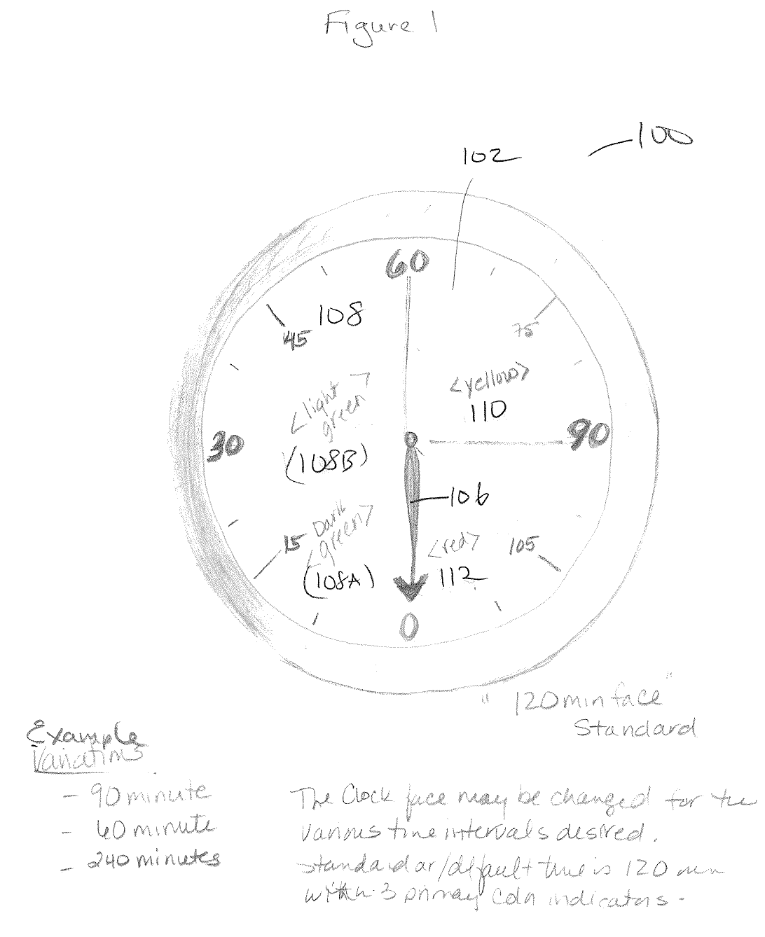

FIG. 2 illustrates a side perspective view of one example of the device.

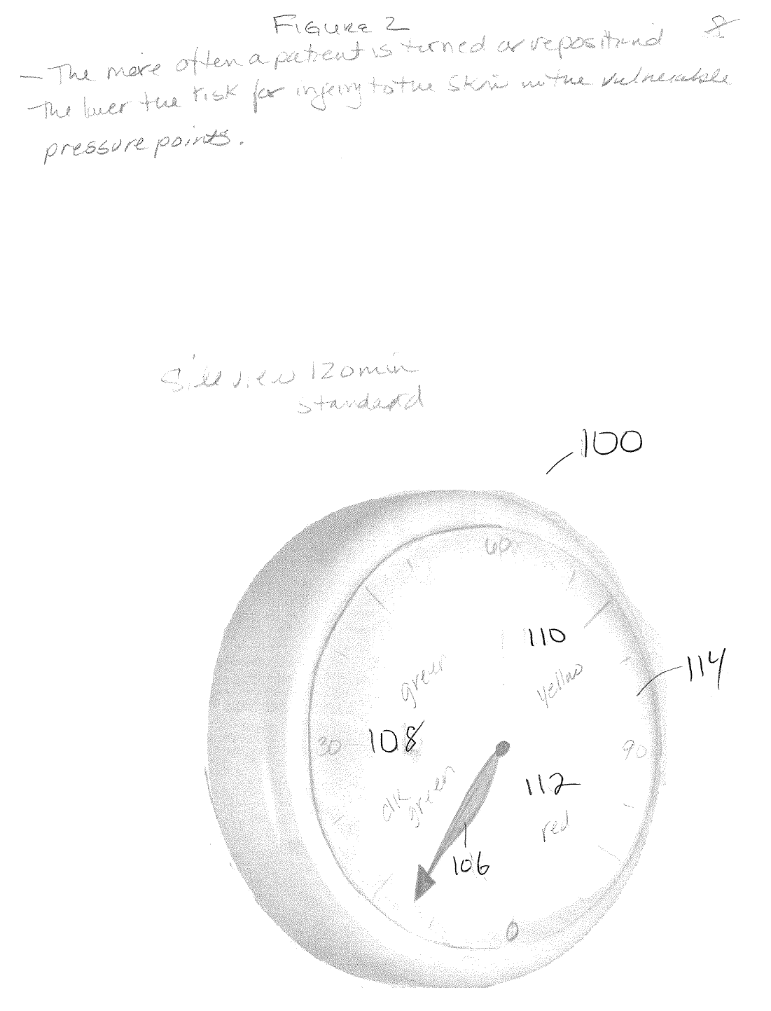

FIG. 3 illustrates an embodiment of the electric control circuit block, which interfaces with the DC motor and lights according to various embodiments of the invention.

FIG. 4 illustrates an exploded view of the major components of the device.

FIG. 5 illustrates an example of the device mounted to the wall in a patient's room and a how a care giver might activate it with a hand.

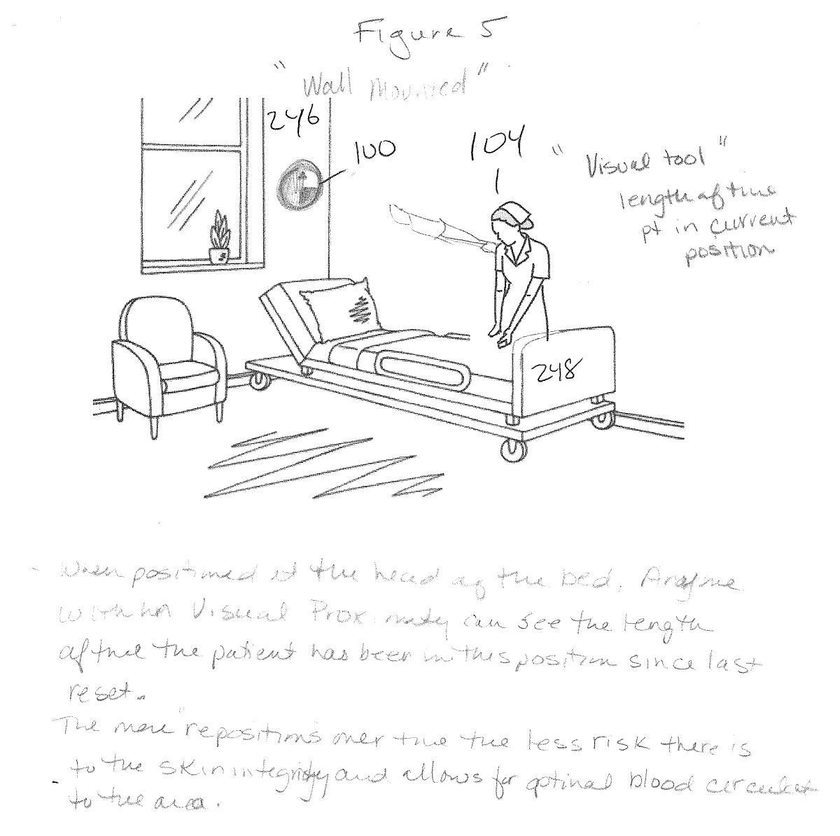

FIG. 6 illustrates an example of the device mounted to the wall in a patient's room and a how a care giver might activate it with an elbow.

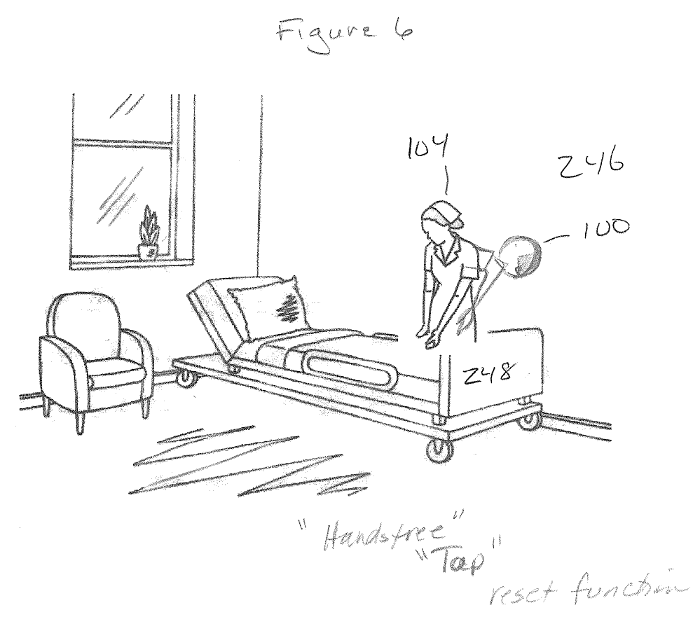

FIG. 7 illustrates an example of the device mounted to footboard on a patient's bed and a how a care giver might activate it with a hand.

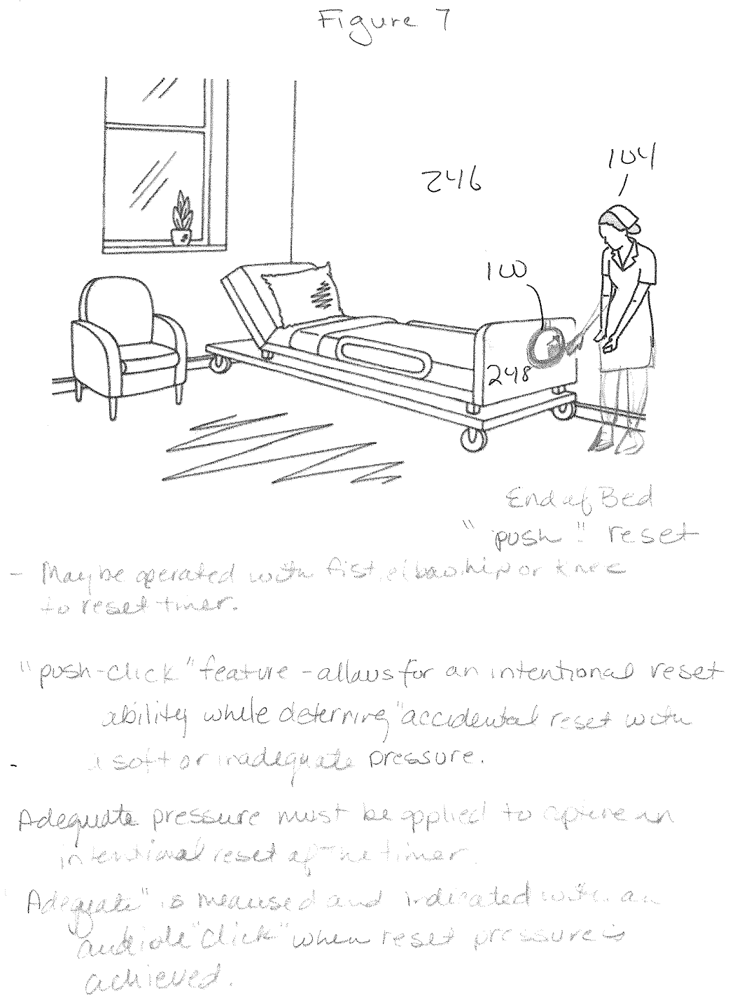

FIG. 8 illustrates an example of the device mounted to footboard on a patient's bed and a how a care giver might activate it with a knee.

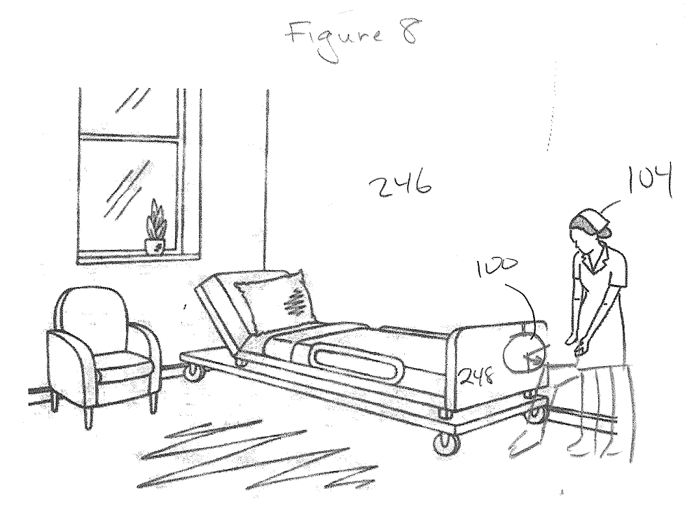

FIG. 9 illustrates an example of the device mounted to rail of a patient's bed and a how a care giver might activate it with a hand.

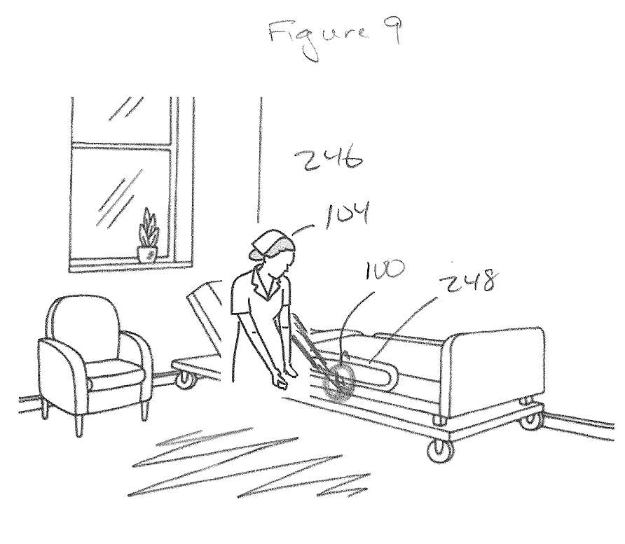

FIG. 10 illustrates an example of the device mounted to the rail of a patient's bed and a how a care giver might activate it with a knee.

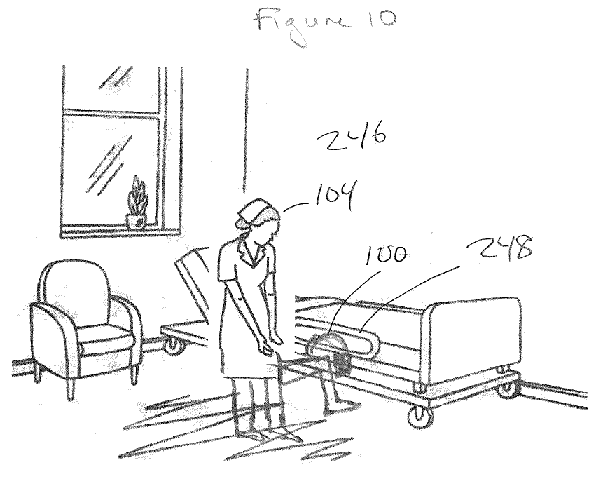

FIG. 11 is a representation of an example device.

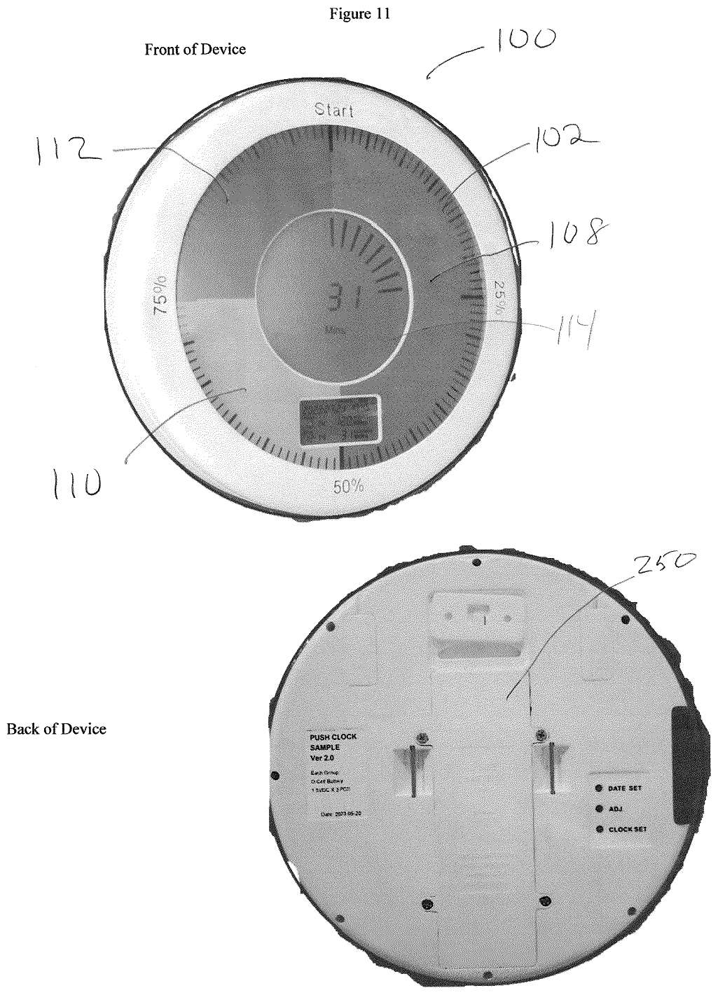

FIG. 12 is a block diagram representation of a method of use for a clock timer to indicate the need to reposition a patient and the like.

Like reference symbols in the various drawings indicate like elements.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

New device to alert caregivers of the need to reposition a patient are discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention, and is not intended to limit the invention to the specific embodiments illustrated by the figures or description below.

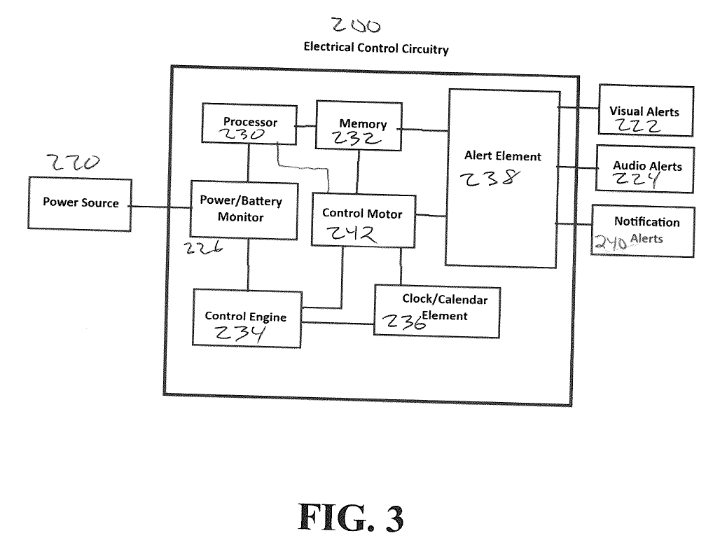

FIG. 3 illustrates an embodiment of an electric control circuit block diagram 200 of a clock/timer 100 configured to monitor and alert a person responsible 104 for providing action to a patient in accordance with the embodiments described herein. The electric controller 200 may be coupled within a clock/timer 100 as described herein, and can be in electrical communication with a power source 220, a plurality of lights 222 (e.g., LEDs, OLEDs, etc.), one or more components of audio 224 (e.g., speakers), etc. The power source 220 may be a battery, which may be monitored by a battery monitor 226. In examples, the battery may include disposable or rechargeable batteries coupled within the clock/timer 100, as described herein. In examples, the power source 220 may include an additional or alternative power source such as a physically, wired power source via a power outlet.

Electrical control circuit 200 can include a processor 230 and a memory 232. In examples, the processor 230 can be a microprocessor, logic circuit, Application-Specific Integrated Circuit (ASIC) state machine, gate array, controller, or the like. The electric control circuit 200 may generally be configured to control the clock/timer 100, such as monitoring a stage or portion (e.g., 108, 110, 112) of a timing mechanism for alerting a responsible person 104 to perform an action on a patient (e.g., reposition the patient, provide scheduled medication, etc.). The electric control circuit 200 may generally be configured to provide alerts via one or more of a combination of constant lights, flashing lights, sound alerts, or the like based on the programmed timing intervals associated with the clock/timer 100, patient requirements, etc.

In examples, programmed parameters, such as the timing intervals, timing of alerts, sounds or other notifications may be preprogrammed or selectively adjusted by a clinician based on particular circumstances of the patient, when action should be taken, etc. Such programmed parameters may be stored in the memory 232 for specific implementation by a control engine 234. A clock/calendar element 236, which may assist in maintaining and monitoring an accurate date/time for the system may also be incorporated within electrical control circuitry 200. In examples, an alarm element 238 can be configured to activate one or more visual alarms/alarms 222, audio alarms/alerts 224, signal notifications 240, or other alarm features. In examples, such alarm features or notifications may include sending messages/signals to a person responsible 104 for action, activating a variation of lighting/illumination features or auditory alerts, etc. to inform a person responsible 104 that action should be taken and provided to a patient.

In examples, a control motor 242 may be in communication with at least one of the processor 230, control engine 234, clock/calendar element 236 to facilitate the tracking and timing of the programmed parameters and mechanical/electrical elements of the clock/timer 100. For example, a person responsible 104 for action may program the clock/timer 100 for a 2-hour time interval. In examples where the clock/timer 100 is a mechanical clock, the control motor 242 may assist in facilitating the display to visualize a time remaining or time until action should be taken, as described in more detail below. In examples, the control motor 242 may include a DC motor to facilitate movement of the components within the clock/timer 100 itself.

It should be understood that while a variety of control mechanisms, alerts, etc. have been exemplified, additional or alternative mechanisms and components are contemplated to facilitate the monitoring of programed timing intervals, alerts, etc. to assist a responsible person to take action with a patient.

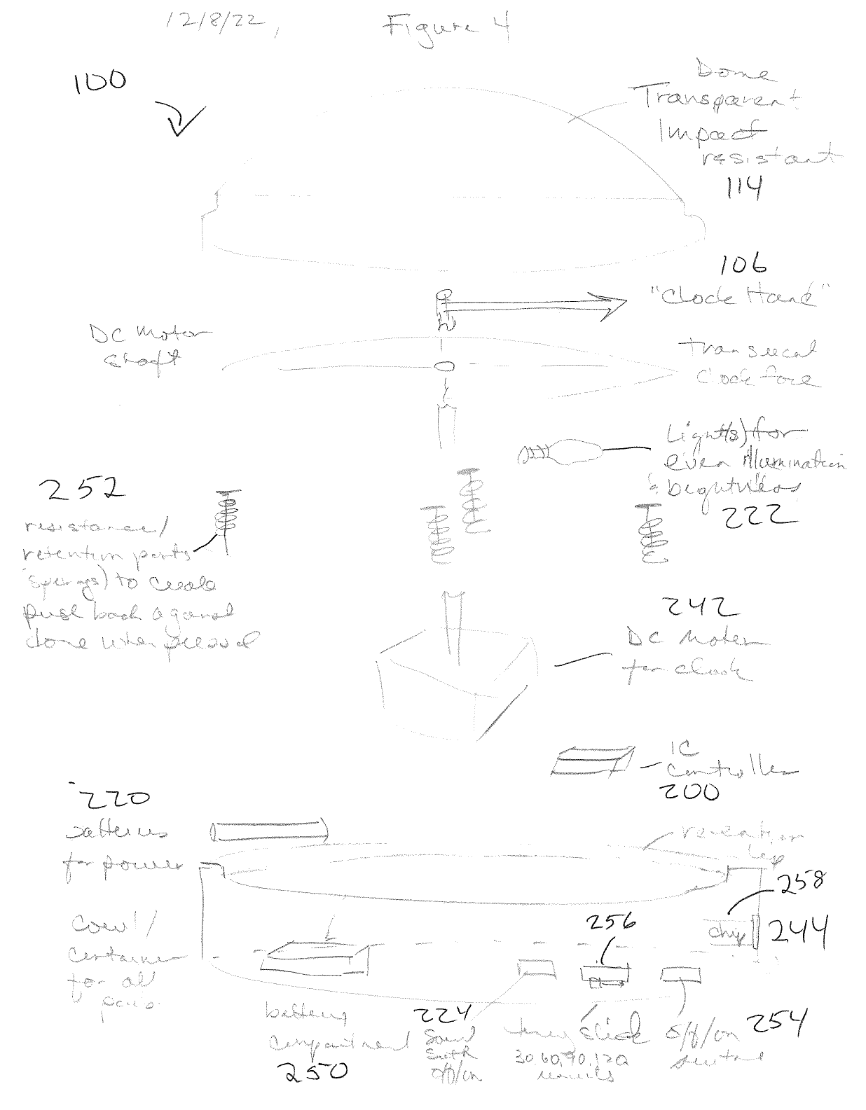

The present invention will now be described by referencing the appended figures representing preferred embodiments. FIG. 4 depicts an exploded perspective view of the elements that comprises a device 100 to signal the need to reposition a patient (“the device”) according to various embodiments of the present invention in preferred embodiments. The device 100 is held within a container which holds all the components depicted but allows for the transparent dome to move when pressed. When the dome 114 is pressed the control circuit block 200 resets the time and moved the timing hand 106 back to the zero or start position and the timer immediately restarts. The user also experiences a tactile response to confirm the reset has occurred. The timer runs until the end of the cycle and stops at the maximum time. As the time progressed from the start time to the maximum time, the lights 222 change in increasing intensity and as the timer moves to the maximum time and stops there (even if the time exceeds the maximum) although actual time between resets is collected. At and after the time is exceeded the lights 222 continue to flash at the highest intensity until the device is reset or until a limit is reached i.e. to preserve battery life. Sound 224 is also an option indicated by the user. Whenever the device 100 is reset, the timer returns to the starting point and timing restarts and the lights 222 return to the initial setting. Resets may also occur via a Bluetooth signal, or other non-tactile means, from a Bluetooth capable device such as a cell phone or tablet, or via a voice signal through a similarly affiliated device.

The device 100 captures data involved in its use and stores it on a removable data chip 244. The data store chip 244 is accessible from the bottom or the side of the device 100. If from the side of the device 100, the device 100 does not need to be removed from the wall 246 or bedside 248. If from the bottom, then, like the batteries, the device 100 will need to be temporarily removed to allow the user to access the chip 244. Data captured includes: time of day, time till each reset, at a minimum.

The device 100 may be battery powered and, in the current embodiment of the device, the batteries 220 are held in a compartment 250 accessible from the underside, side, front, etc. of the device 100. In examples, the batteries 220, or alternative power source may be configured to power the device 100. In examples, such alternative power source may be a cable or other wiring in electrical connection to the device and a power source, such as an outlet. When the batteries 220 are low, the lights 222, or a symbol, flash in a distinct pattern alerting the user that they need to be replaced.

A resistance mechanism 252, such as the spring on posts depicted in FIG. 4 provides a tactile resistance to pressure applied to reset the timer. In addition, a noticeable “click” is felt through the device to the user confirming that the reset has occurred without looking at the device 100.

The control circuit block 200 provides for electronic management of the timer and therefore the hands and the lights seen by the caregiver 104, communications such as Bluetooth and voice commands, and storage of data. Also affiliated with the control circuit block is the off/on switch 254, a timer slide/switch 256 with toggles between total time e.g. 120 minutes, 4 hours, 90 minutes, 60 minutes, etc., the data chip receptacle 258, and the battery compartment 250.

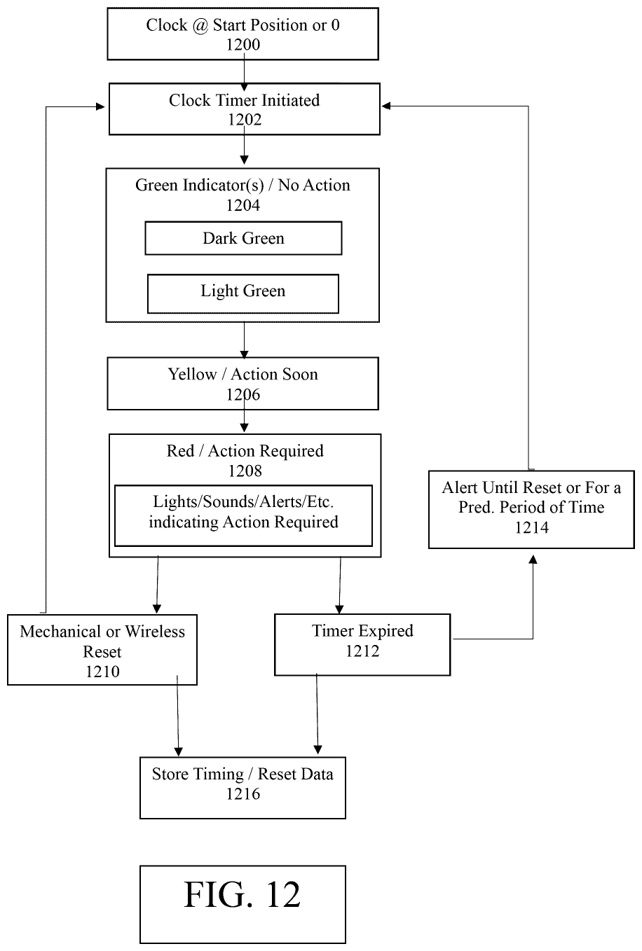

FIG. 12 is a block diagram representation of a method of use for a clock/timer 100 to indicate the need to reposition a patient and the like. At 1200, the clock/timer 100 may be set to a ‘zero’ position. In examples, alternative positions may be set such that the clock/timer 100 initiates at a time alternative to ‘zero’ (i.e. a start position) depending on the clock/timer face intervals and the amount of time set to monitor, count, etc.

At 1202, the clock/timer 100 may be initiated or automatically begun again, such as in a repeating cycle (as described below).

At 1204, the clock/timer 102 face may be portioned into a plurality of sections. For example, in the initial stages, where no action to be taken is necessary, the first quarter, half, or section (e.g., 108, 108A, 108B) of the clock/timer face 102 may display a green hue or color. In examples, the first half or portion 108 of the clock timer face 102 may be colored with a particular color, such as green, indicating no action is necessary within that colored half or portion of the clock timer face 102. In examples, the first half or portion 108 of the clock timer face 102 may be portioned into additional sections, such as quarters (e.g., 108A, 108B). In examples, a first quarter or initial portion 108A of the clock timer face 102 may be colored with dark green color or hue, which may indicate a timer has just begun. Following the dark green color, an additional second or next section 108B of the first half or portion of the clock timer face 102 may include a lighter green color, which may indicate to a user that time is still passing, but still no action is yet necessary. In examples, the first quarter, half or section (108, 108A, 108B) of the clock timer face 102 may display a single, solid color, such as a single shade of green. In examples, other colors are contemplated to demonstrate the clock timer has been reset, has begun, or indicate initial timing stages, prior to action from a clinician.

At 1206, a preceding quarter or portion 110 of the clock timer face 102 may display a yellow color or hue. In examples, a transition into the alternative, yellow color may equate to yet a third quarter of the clock timer display 102 and signify that approximately half of the time has elapsed before action is required. For example, the yellow display may represent that action is required soon and a clinician or caretaker 104 should prepare accordingly.

At 1208, the clock timer face 102 may have a final fourth quarter or portion 112 of the clock face 102 that displays in a red color. In examples, when the clock timer reaches the red portion of the display 102, this may signify to a clinician 104 that action is required. In examples, any variety of indicators, such as lights 222 (e.g., LEDs), speakers 224, alerts, sounds, alarms, etc. may be incorporated into the clock timer 100 such that, when action is required, at least one or more of the indicators may activate. Thus, alerting a clinician 104 that action is required, whether visually 222, audibly 224, transmitting a signal notification 240, etc.

At 1210, if clinician 104 notes the indicators in time, the clinician 104 can perform the action, for example, by repositioning the respective patient. Once completed, the clinician 104 may manually or mechanically reset the clock timer 100, for example, by touching or pressing the clock timer face 102 (e.g., a transparent dome 114). In examples, the clock timer may be in communication with a wired or wireless device (e.g., Bluetooth), such that the clinician may reset the clock timer wirelessly. Once reset, the clock timer 100 may be reset back to ‘zero’ or a predetermined start position, as described herein, and automatically initiate the clock timer at 1202. Thus, the cycle between, at least, 1202, 1204, 1206, 1208, and 1210 may be repeated.

At 1212, a clinician 104 may not hear or see the timer clock indicators, or may be too busy (e.g., taking care of another patient) to perform an action corresponding to the clock timer. In examples, at 1214, when time has expired, an additional alert or notification may be activated, or the alerts and notifications may continue. In examples, alerts or notifications may include various sound indications, flashing lights, wireless signals sent to remote devices (e.g., cell phones, tablets, external computers, base station, etc.). It should be understood that, while exemplary alerts and notifications have been presented alternative and additional alerts and notifications are contemplated, which may gain the attention of a clinician, someone in charge of taking care of a patient, or other users, including the patent themselves. Indications may continue for a predetermined period of time or until the clock timer is finally reset. In examples, should a clinician not reset the clock timer after the predetermined period of time, a default may reset the clock timer back to a ‘zero’ or start position and automatically reinitiate the clock timer at 1202.

At 1216, timing and reset data may be stored for future use and evaluation. Records and logs can be used to keep track and monitor frequency and timing of when a clinician reset the clock timer and how often and how long the clock timer has been left to elapse.

In examples, pending actions taken or not taken by the clinician, repeating cycles may be completed between 1202, 1204, 1206, 1208, 1210, 1212, 1214, and 1216.

It should be understood that while green, yellow, and red colors are depicted herein, any variation of colors, indicators, etc. are contemplated.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

1. A device to alert a responsible person to take action to service or provide a service to a specific patient, the device comprising:
(a) a programmable block into which, at a minimum, a date and a time is entered and a desired time interval is chosen, the desired time interval defining a time until alert, and a time between activation of the device or a most recent reset and a next reset made is tracked;
(b) a button which activates or resets the device; and
(c) a display which presents both an amount, graphically or quantitatively, of time elapsed and a time until the time interval is exceeded.

2. The device of claim 1, further comprising the programmable block allows for an input of a current date and a current time and allows for the time until alert to be adjusted to a user's needs including selectively adjustable between at least one or more of about 30 minutes, 2 hours, and 6 hours.

3. The device of claim 1, further comprising a removable data storage device which captures and stores the captured date, time, time of start and time of stop; and wherein the removable data storage device calculates an interval between each start and stop time and allows access electronically or by virtue of being removed so such data may be analyzed.

4. The device of claim 1, further comprising a color coded face to indicate to a viewer and recognize how much time has elapsed since a last action to service or provide a service to a specific patient or how much time is left before the action becomes too late.

5. The device of claim 1, further comprising a selectable surface that, when touched or depressed, resets the device to start and automatically restarts the time until alert.

6. A device to alert a responsible person to take an action to service or provide a service to a specific patient, the device comprising:
(a) a programmable block into which, allows for entry of a current date and a current time and a desired time interval to track upon initial set up, the desired time interval defining a time until alert;
(b) a button in the form of a dome that covers a face of the device, which may be activated with a push from a user;
(c) a multi-color and digital display which presents both an amount of time elapsed and a time until the time interval is exceeded.

7. The device of claim 6, wherein the programmable block allows for the input of the current date and time and allows for the time until an alert to be adjusted to the user's needs, wherein the time until the alert includes at least one of about 30 minutes, 1 hour, 2 hours, 4 hours, 6 hours, 8 hours, 12 hours, 1 day.

8. The device of claim 6, further comprising a removable data storage card which captures and stores the captured current date, current time, time of start and time of stop, and may calculate an interval between each start and stop time and allows access by virtue of being removed so such data may be analyzed, wherein the data being displayed on the face of the device.

9. The device of claim 6, further comprising a color coded face with a green zone in a first section of a face of the device in which the time indicator progresses, a yellow zone in a second section of the face, and a red zone in a third section of the face, which enables a viewer to readily recognize an amount of time that has elapsed since a last action to service or provide a service to a specific patient or an amount of is left before the action becomes too late.

10. The apparatus of claim 8, further comprising a display in the center of the device which shows a progression of time through the zones with an elapsed number of minutes displayed in the center of the device.

11. The apparatus of claim 8, further comprising a display on the device which shows the date, time, start and the time at the maximum interval time, the elapsed time, and a battery power indicator.

12. The device of claim 6, further comprising a surface that covers the entire face in the form of a clear dome that, when touched or depressed, resets the device to start and automatically restarts the time.

13. The device of claim 6, further comprising an alert in the form of a red flashing light that begins when the interval time has been exceeded a predetermined time period and remains flashing for a specific amount of time or until the button has been pressed.

14. A method for providing a visual, auditory, or alternative form of communication to alert a responsible party to take action to service a patient using an alert device, the method comprising:
(a) capturing current date and time information, choosing a maximum interval between events, wherein the maximum interval between events defines a time until alert, and calculating a time between resets of the device;
(b) activating or resetting the device to mark an end of a previous interval and a start of a next interval; and
(c) displaying an elapsed time and a time until a maximum interval is exceeded.

15. The method of claim 14, comprising the input of the current date and time information and allow for the time until alert to be adjusted to a user's needs, wherein the time until alert includes at least one of 30 minutes, 1 hour, 2 hours, 4 hours, 6 hours, 8 hours, 12 hours, 1 day.

16. The method of claim 14, comprising capturing and storing the captured date, time, time of start and time of stop, and calculating the interval between each start and stop time and allowing access by virtue of being removed, such that the captured data may be analyzed.

17. The method of claim 14, comprising a color coded face to provide a viewer an easily recognizable display for how much time has elapsed since the last action or how much time is left before the action becomes an interval that has been exceeded.

18. The method of claim 14, comprising a display which shows the progression of time through one or more zones of the display with the elapsed number of minutes in the center.

19. The method of claim 14, comprising a display on the device which shows the date, time, start and the time at the maximum interval time, the elapsed time, and a battery power indicator.

20. The method of claim 14, comprising a surface that when touched or depressed, resets the device to start and automatically restarts the time.
